(19) United States
(12) Patent Application Publication
(10) Pub. No.: US 2007/0141605 A1
(43) Pub. Date: Jun. 21, 2007

(54) Portable preparation, analysis, and detection apparatus for nucleic acid processing

(75) Inventors: Charles S. Vann, El Granada, CA (US); Michael Greenstein, Los Altos, CA (US); Yuh-Min Chiang, Foster City, CA (US)

Correspondence Address: KILYK & BOWERSOX, P.L.L.C., 3603 CHAIN BRIDGE ROAD, SUITE E, FAIRFAX, VA, 22030, US

(73) Assignee: Applera Corporation, Foster City, CA (US)

(21) Appl. No.: 11/602,149

(22) Filed: Nov. 20, 2006

Related U.S. Application Data
(60) Provisional application No. 60/738,589, filed on Nov. 21, 2005.

(51) Int. Cl. C12Q 1/68; C12M 3/00

(52) U.S. Cl. 435/6; 435/287.2

(57) ABSTRACT

The present teachings comprise a device and method for lysing and/or purifying biological sample. The device can comprise a cartridge having a chamber containing a biological sample receiving region, a plurality of electrodes, and one or more sieving matrices. The electrodes can be configured to lyse the biological sample through the production of a pulsed electrical field. The electrodes can also be configured to heat lyse the biological sample. The electrodes can also be configured to electrophoretically move the biological sample through one or more sieving matrices. A portion of the sample can be isolated on a membrane. The portion of the sample isolated on the membrane can be amplified and detected. A portion of the sample can be isolated in a collection area present in the cartridge. The portion of the sample isolated in the collection area can be removed from the cartridge.

### CROSS-REFERENCE TO RELATED APPLICATIONS

The present application claims the benefit of earlier filed U.S. Provisional Patent Application No. 60/738,589, filed Nov. 21, 2005, which is incorporated herein in its entirety by reference.

### FIELD

Various embodiments of the present teachings relate to devices and methods for the preparation and/or purification of biological materials.

### INTRODUCTION

Preparing a biological material for nucleic acid analysis has, in the past, required complex and expensive devices. For example, in some described methods a biological material is collected and deposited in a sonicator for cellular lysis. After lysis, the sample is purified in a separate device, for example, a centrifuge. From the centrifuge the sample is transferred to a water bath or other suitable thermal cycling device for nucleic acid amplification and sequence detection. Finally, amplified nucleic acids can be sequenced using a slab-gel or capillary electrophoresis device. There exists a need for a single device that can accomplish one or more of these tasks.

### SUMMARY

The present teachings relate to a device, system, and method for processing biological samples. The device can comprise a cartridge. The cartridge can comprise a chamber. Two or more electrodes can be disposed inside the chamber. One or more sieving matrices can be disposed between the electrodes inside the chamber. The chamber can comprise a sample receiving area adjacent one of the electrodes. The chamber can comprise a collection area adjacent the sieving matrix. In some embodiments, a capture membrane can be disposed inside the chamber adjacent the sieving matrix.

The cartridge can comprise a buffer solution. The buffer solution can be electrically conductive. Nucleic acid amplification reactants can be loaded in the buffer solution. The nucleic acid amplification reactants can comprise, for example, primers and or probes designed for the detection of one or more specific nucleic acid sequences. The nucleic acid amplification reactants can comprise reporter molecules, for example, fluorescer/quencher molecules as are known in the art.

According to some embodiments, the cartridge can comprise a cap. The cap can comprise one or more electrodes. The cap can comprise a collection device, for example, a scoop, stick, needle, swab, or the like. The cap can comprise electrodes configured to electroporate cells and/or viruses for, for example, to irreversibly electroporate cells and/or viruses.

According to various embodiments, a system for preparing and/or purifying a biological sample can comprise a chamber adapted to receive the cartridge. The system can comprise electrical connections. The electrical connections can connect the electrodes of the cartridge to a power source. The system can comprise a control unit. The control unit can be electrically connected to the electrical connections and/or the power source.

According to various embodiments, the system can comprise a capacitor. The capacitor can be electrically connected to one or more of the electrodes. The capacitor can be controlled by a control unit. The system can comprise a resistor. The resistor can be electrically connected to one or more of the electrodes. The resistor can be controlled by a control unit.

According to various embodiments, a method of preparing or purifying a biological sample can comprise introducing the biological sample into a sample receiving region in the cartridge. The biological sample can be lysed mechanically or through irreversible electroporation. A portion of the biological sample having a net electric charge can be electrophoretically moved through the sieving matrix. Electrophoretic motion of a portion of the biological sample can result from the creation of an electric field gradient between electrodes present in the cartridge. For example, an electric field gradient can be provided of sufficient force to cause nucleic acids to be isolated or separated from proteins and/or other cellular debris. According to various embodiments, proteins can be isolated from nucleic acids.

A desired portion of the biological sample can be separated from an undesired portion through a manipulation of the polarity and/or strength of an electric field gradient formed by the electrodes. For example, the voltage or polarity of the electric field can be pulsed. Eletrophoretic motion can cause a portion of the biological sample to emerge from the sieving matrix. The portion of biological sample moved in this manner can be removed from the cartridge.

According to various embodiments, nucleic acid disposed in a biological sample can be captured in a nucleic acid capture membrane. A portion of the biological sample, for example, nucleic acids, that emerges from the sieving matrix can be captured on a capture membrane. The captured portion of the sample can be amplified on the membrane. The nucleic acid or an amplification product thereof can be detected. The nucleic acid can be electrophoresed into pores of the nucleic acid capture membrane, for example, such that the nucleic acid can be captured on a wall of the pore. Nucleic acid amplification reactants can be present in the cartridge, for example, PCR reagents can be pre-loaded or pre-deposited in the cartridge. PCR reagents can freely flow inside or through the membrane. For example, a Taqman probe can be cleaved to release reporters during PCR of a target that reacts with the Taqman probe. During the thermal cycling, the heat/cool cycle can unquench reporters when PCR reagents react with a target.

The amplified sample portion can be detected by, for example, the detection of fluorescent probes incorporated into amplified nucleic acids present on the membrane. The membrane can be illuminated with a light source, for example, a light-emitting diode, a laser, or a lamp. The probes or reporters can absorb a first wavelength range of radiation the illumination-light and emit radiation at a different wavelength of light (fluorescence). The emission light can be detected or visually inspected through the window.

It is to be understood that both the foregoing general description and the following detailed description are exemplary and explanatory only. The accompanying drawings, which are incorporated in and constitute a part of this application, illustrate several exemplary embodiments and together with the instant description, serve to explain the principles of the present teachings.

### DRAWINGS

The skilled artisan will understand that the drawings described below, are for illustration purposes only. The drawings are not intended to limit the scope of the present teachings in any way.

FIG. 1 is a cross sectional view of a cartridge in electrical communication with a power source, according to various embodiments;

FIG. 2A is a perspective view of a cartridge processing device, a cartridge, a biological sample swab, and a quarter;

FIG. 2B is a cross sectional view of the cartridge processing device of FIG. 2A. according to various embodiments;

FIG. 2C is a top plan view of a filter wheel;

FIG. 3A is a cross sectional view of a cartridge according to various embodiments;

FIG. 3B is a plan view of electrodes 216a-216e shown in FIG. 3A, when viewed from line 3B-3B in FIG. 3A;

FIG. 3C illustrates various steps involved with of utilizing the cartridge of FIG. 3A;

FIG. 4 is an electrical schematic diagram of a system adapted to process a cartridge according to various embodiments;

FIG. 5A is a sectional view of a cartridge according to various embodiments;

FIG. 5B illustrates various steps involved with of utilizing the cartridge of FIG. 5A;

FIG. 6 is an electrical schematic diagram of a system according to various embodiments;

FIG. 7 is a sectional view of a cartridge according to various embodiments;

FIG. 8A is a sectional view of a cartridge according to various embodiments;

FIG. 8B is a plan view of electrode 814 shown in FIG. 8A, when viewed from line 8B-8B of FIG. 8A.

### DESCRIPTION

According to various embodiments, the present teachings describe a cartridge that can enable the isolation and/or detection of biological materials, for example, nucleic acids, proteins, cells, or viruses. The biological samples can be prepared and detected within a single, self-contained cartridge comprising pre-loaded reagents, and without motors, valves, or sensors. Assay detection can be performed optically through a transparent wall of the cartridge. One detection scheme involves use of a human eye as the detector, however, other means of detection can be used, for example, a camera or scanning photo detector.

According to various embodiments, a human eye can be used to detect fluorescence. The human eye can detect as few as 10 photons landing within a 10 arc minute diameter at the back of the eye (about 50-micrometer diameter). Bunching the photons in a tighter circle does not reduce the number of photons detectable. The experimental conditions for this level of detection included the following in a study by Hecth, Schlaer and Pirenne: eye dark adapted for 40 minutes; left eye occluded, right eye only tested; eye fixated a very faint dim red light; test spot located 20° nasal to fixation; test spot diameter was 10 arc minutes; test light was flashed for 1 millisecond; and wavelength was 510 nm (green).

In some embodiments, nucleic acid detection can be performed optically through a transparent window of the cartridge. One detection method can comprise visual inspection of the reactions within the cartridge. In some embodiments, other means of detection can be used. The system cartridge can comprise a low cost, hand-held, micro device. The system can use real time PCR chemistry. The system can be used to detect different types of viruses for example, HIV.

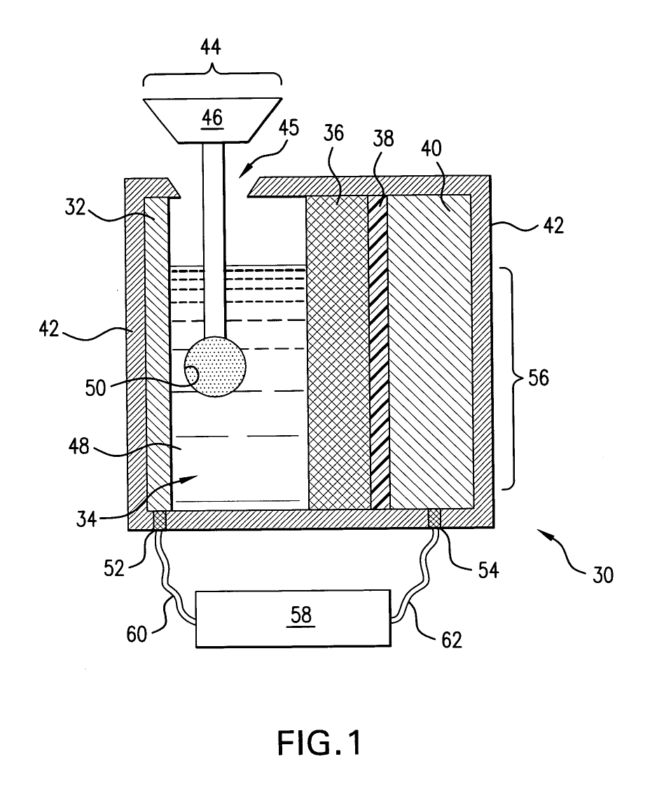

According to various embodiments, and as illustrated in FIG. 1, cartridge 30 can comprise a chamber 42. Any chamber described in the present application can be made of any suitable material, for example, plastic or glass. A chamber can be non-conductive to electricity. A chamber can be transparent to light. Chamber 42 can comprise a window or transparent portion 56. In other embodiments, the entire chamber can be transparent.

One or more electrodes can be disposed inside cartridge 30. Any electrode described in the present application can comprise a single electrode, or can comprise a plurality of electrodes. An electrode can comprise a conductive material. An electrode can comprise a metal that does not corrode in or react with an aqueous solution. An electrode can comprise, for example, palladium, platinum, gold, or indium tin oxide. Other materials capable of conducting electricity can be utilized as electrodes. An electrode can be configured to be transparent to light, for example, the electrode can comprise a mesh or the electrode can comprise a sputter deposited layer deposited on a transparent support. An electrode transparent to light can comprise indium tin oxide.

The electrodes can be disposed closely spaced. The closely spaced-apart electrodes can be used to produce relatively large electrical fields without utilizing large voltage differentials between the electrodes. For example, two electrodes can be disposed about 600 μm from one another. A low voltage, for example, about 2.6V, available from an AA-size battery can produce an electrical field having a field strength of about 43 V/cm⁻¹ between the two electrodes. Voltage provided to the electrodes can be about one Volt or greater, for example, about 5 Volts or greater, or about 10 Volts or greater. The electrodes can be utilized to perform various operations, for example, electrolysis, electroporation, electro-osmosis, or electrical kinetic movement of polarized analytes in a sample. When an operation that produces a gas as a by-product, for example, electrolysis, a gas-porous material that is impervious to liquids, for example, PDMS, can be disposed in the cartridge to vent the gas.

According to various embodiments, when an electrical field is generated and the electrodes are in contact with water molecules, the electrical field can be used to generate hydroxide (OH⁻) at the cathode (negative electrode) and hydrogen at the anode (positive electrode). The water molecules can be provided by biological samples and/or an aqueous buffer or electrolyte. Excessive hydroxide is known to cleave the fatty acid-glycerol ester bonds in phospholipids molecules, resulting in the production of fatty acid chains and lysophospholipids. At certain concentrations of hydroxide, for example, about 20 mM to about 100 mM, and at certain pH levels, for example, about 11.2 to about 12.55, these effects can be observed in lysed red blood cells in less than about 100 seconds. In various embodiments, in the absence of an electrical field, the hydroxide and hydrogen can turn to water when mixed. The water so produced can eliminate the need to wash a sample after lysing. The advantages of fast, low-voltage lysing, and no washing after lysing are attractive for portable devices.

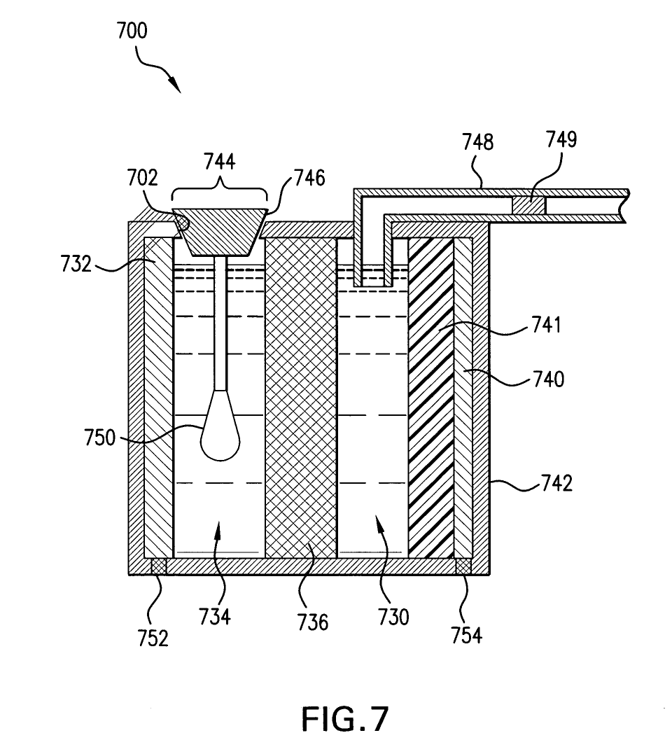

In FIG. 1, electrodes 32 and 40 of cartridge 30 can be separated by several millimeters, for example, from about 1 mm to 50 mm, or about 6 mm. An increase in a separation distance between the two electrodes can require an increase in the voltage applied to the two electrodes, for example, enough voltage to generate a field strength of about 43 V/cm⁻¹ between the two electrodes. For example, at about 6 mm separation between electrodes, a 26V voltage can provide a field strength of about 43 V/cm⁻¹. The voltage applied at the two electrodes can be proportional to the distance between the two electrodes. Similar distances and voltages can be used for cartridge 700 of FIG. 7.

According to various embodiments, a first electrode 32 can be disposed adjacent to a first end inside cartridge 30. A second electrode 40 can be disposed adjacent to a second end inside cartridge 30. The first and second electrodes 32 and 40 can be electrically connected to contacts 52 and 54 respectively. Contacts 52 and 54 can provide a connection to electrical leads 60 and 62, respectively, and an external power source 58. Power source 58 can comprise, for example, a battery, a transformer connected to alternating current, or a power supply adapted to provide a pulse emission current and/or a direct current as desired.

According to some embodiments, a sieving matrix 36 can be disposed inside the cartridge dividing cartridge 30 into two sections. Sieving matrix 36 can be disposed within cartridge 30 such that particles disposed in cartridge 30 cannot freely move from one section to the other section without passing through sieving matrix 36.

A sieving matrix, as described in the present application can comprise, for example, a micro-porous filter, a frit layer, a bead layer, a fiber composite layer, a laser drilled membrane, or any other material that selectively allows nucleic acids to pass through the sieving matrix. A sieving matrix can have apertures of, for example, one micron or less. The sieving matrix can allow different molecules, to be moved by a force, for example, an electric field or a pump to migrate the molecules through the sieving matrix at differing rates. Migration rates differ depending on the size, shape, or charge of the migrating molecule. For example, smaller linear molecules can pass through the sieving matrix more quickly than larger or highly branched molecules due to interactions of the molecules with the sieving matrix itself. The sieving matrix can be essentially impermeable to larger molecules, for example, complex cellular debris and/or organelles. The sieving matrix can function to trap some molecules, for example, proteins, while permitting other molecules to pass through the matrix.

According to various embodiments, a capture membrane 38 can be disposed inside cartridge 30. Capture membrane 38 can be disposed adjacent to sieving matrix 36.

A capture membrane as described in the present teachings can be a material having pores ranging in sizes of about 5 nm, 4 nm, 3 nm, 2 nm, 1 nm, 0.5 nm, 0.25 nm, or the like. A capture membrane can be a specific sequestering agent. The capture membrane can form an association with nucleic acid molecules. The capture membrane can sequester large molecules of DNA (about 100 base pairs or greater), but can allow smaller molecules such as probes, primers and single nucleotides to freely diffuse through the membrane without being sequestered. The capture membrane can comprise, for example, an Anopore® membrane from Whatman® Inc., Florham Park, N.J., or any other suitable nucleic acid specific sequestering agent known to one skilled in the art.

Cartridge 30 can comprise an opening or aperture 45. Aperture 45 can provide access to a sample receiving space 34 inside cartridge 30. Sample receiving space 34 can be defined on one side by first electrode 32, and on another side by sieving matrix 36.

According to various embodiments, the present teachings can comprise a collection device 44. Collection device 44 can comprise a cap 46. The cap can be configured to seal aperture 45. Collection device 44 can comprise a sample collector 50, for collecting biological samples. A sample collector described in the present teachings can comprise a swab, a spoon, an aspirator, a needle, a syringe, or any other suitable collection device known by one skilled in the art.

According to various embodiments, a biological sample can be collected by collection device 44 and inserted into sample receiving space 34. Sample receiving space 34 can be sealed by cap 46. A buffer 48, for example, an electrophoresis buffer, can be loaded or preloaded into cartridge 30. Buffer 48 can suspend a biological sample. A biological sample described in the present teachings can comprise, for example, any type of intact or lysed biological cell or virus and/or component parts thereof. For example, the biological sample can comprise DNA, RNA, proteins, or the like. Biological samples can comprise, for example, blood, fecal matter, sputum, saliva, urine, mucus, tissue samples, or the like.

Intact cells or viruses in sample receiving space 34 can be lysed. Lysis can occur by heating the biological sample. Lysis can be performed using electrolysis. First electrode 32 can be configured to function as a resistive heater. Applying a voltage to first electrode 32 can cause the electrode to heat up and thereby heat sample receiving space 34. Sample receiving space 34 can be heated to a temperature sufficient to lyse biological materials, for example, to a temperature from about 96° C. to about 99° C.

In operation an electric charge can be applied to first electrode 32. An opposite electric charge can be applied to second electrode 40. In this way, an electric field gradient can be created between the first and second electrodes. The field gradient can attract or repel molecules in the sample receiving area depending on the charge of the molecules. For example, a positive charge on the second electrode will attract negatively charged molecules, for example, nucleic acids. The charges on the electrodes can be reversed and/or the voltage applied to the electrodes can be altered according to characteristics of the biological sample being purified.

The movement of a portion of biological sample due to the electric field gradient can cause a portion of the biological sample to become associated with capture membrane 38. For example, nucleic acids from the biological sample can become associated with capture membrane 38. Some molecules, for example, cellular debris will be prevented from contacting capture membrane 38 by sieving matrix 36. Nucleic acid amplification reactants, for example, primers, probes, and enzymes can be present in buffer 48. The primers and probes can comprise molecules too small to become associated with the membrane. Probes and primers can freely diffuse through the membrane.

In some embodiments, during operation, the charges applied to first electrode 32 and second electrode 40 can be reversed such that any small moieties of the sample that have migrated adjacent or close to second electrode 40 can be moved back through capture membrane 38, sieving matrix 36, and or sample receiving space 34 toward first electrode 32. The polarity reversal can prevent small moieties present in the sample from inhibiting or otherwise interfering with nucleic acid amplification and/or detection.

Thermal cycling the cartridge with the nucleic acid amplification reactants present in the buffer solution can result in amplification of nucleic acids present on the membrane or in the cartridge. Resistive heating of the electrodes can produce the necessary heat for thermal cycling. First electrode 32 can receive an electric current sufficient to create a temperature from about 60° C. to about 65° C., while second electrode 40 can receive an electric current sufficient to create a temperature from about 90° C. to about 95° C. Second electrode 40 positioned adjacent to capture membrane 38 can quickly heat capture membrane 38 from about 90° C. to about 95 C. First electrode 32 can maintain the remainder of cartridge 30 at a constant temperature of about 60° C. to about 65° C. The actual temperatures contemplated in the present teachings can be modified depending on biological sample to be analyzed and the results desired.

According to various embodiments, the cartridge can be loaded or pre-loaded with nucleic acid amplification reactants. The nucleic acid amplification reactants can comprise probes, primers, and polymerizing enzymes, for example, TaqMan® reagents (Applied Biosystems, Cal.). The reagents are also described in U.S. Pat. No. 6,154,707 to Livak, et al., incorporated herein in its entirety by reference. Other related methods known to one of skill in the art can also be used as deemed appropriate. Such reagents can be used in methods of analyzing nucleic acids.

According to various embodiments, an enzyme that polymerizes nucleotide triphosphates into amplified fragments can comprise heat-resistant DNA polymerases known in the art. Polymerases that can be used comprise DNA polymerases from organisms such as Thermus aquaticus, Thermus thermophilus, Thermococcus litoralis, Bacillus stearothermophilus, Thermotoga maritime, and Pyrococcus ssp. The enzyme can be isolated from source bacteria, produced by recombinant DNA technology or purchased from commercial sources. Exemplary DNA polymerases that can be used include those available from Applied Biosystems (Foster City, Calif.), for example, AmpliTaq Gold™ DNA polymerase; AmpliTaq™ DNA Polymerase; Stoffel fragment; rTth DNA Polymerase; and rTth DNA Polymerase XL. Other suitable polymerases that can be used include, but are not limited to, Tne, Bst DNA polymerase large fragment from Bacillus stearothermophilus, Vent and Vent Exo- from Thermococcus litoralis, Tma from Thermotoga maritima, Deep Vent and Deep Vent Exo- and Pfu from Pyrococcus, and mutants, variants and derivatives of the foregoing. For further discussion of polymerases, and applicable molecular biology procedures generally, see, Ausubel et al., Current Protocols in Molecular Biology, John Wiley & Sons, New York, 2001, and The Polymerase Chain Reaction, Mullis, K. B., F. Ferre, and R. A. Gibbs, Eds., Molecular Cloning: A Laboratory Manual, (3rd ed.) Sambrook, J. & D. Russell, Eds. Cold Spring Harbor Laboratory Press, Cold Spring Harbor, N.Y. (2001), all of which are incorporated herein in their entireties by reference.

According to various embodiments, a fluorophore-labeled probe can bind or be incorporated into a nucleic acid molecule and fluorescence can be detected. The incorporation can result from an enzymatic reaction, or the probe can intercalated into the nucleic acid molecule.

According to various embodiments, a detection system that can comprise one or more excitation sources, at least one detector, and a set of dyes. The excitation sources can be adapted to emit a plurality of different individual excitation beam wavelength ranges, wherein each excitation source emits at least one wavelength that is not emitted in the excitation wavelength range of at least one other of the excitation sources. Each excitation source can comprise a respective individual radiation source or two or more excitation sources can comprise the same radiation source. For example, each excitation source can comprise a separate light-emitting diode (LED) or laser source, or two or more excitation sources can comprise a common broad spectrum light source and appropriate optics, filters, gratings, or the like.

According to various embodiments, a first excitation source can be provided that is adapted to emit a first excitation wavelength range of from about 460 nm to about 475 nm, and a second excitation source can be provided that is adapted to emit a second excitation wavelength range of from about 480 nm to about 495 nm. The second excitation beam wavelength range can be emitted at a different time or in a different direction than the first excitation beam wavelength range. In some embodiments, a group of excitation sources is provided that is adapted to emit two, three, four, or more, different and non-overlapping excitation beam wavelength ranges. U.S. Patent application No. 60/677,233 filed May 3, 2005 contains additional disclosure of suitable fluorophores and is incorporated herein, in its entirety, by reference.

Amplification reaction times, temperatures, and cycle numbers can be varied to optimize a particular reaction. Addition of additives to reduce stutter and reduce non-specific amplification can also be used as determined appropriate by one of skill in the art, for example, see US Patent Application Publication 2005/0112591 to Dimoski et al., which is incorporated herein in its entirety by reference.

According to various embodiments, a method for complete analysis of cells and/or viruses. The method can comprise both sample preparation and analysis can comprise the following from preparation to analysis. A crude sample can be collected with a collection device, for example, a scoop, a swab, or a syringe. The collection device can be inserted into the cartridge. PCR reagents, including probes, primers, enzymes, and buffer can be pre-packaged in the cartridge. Packaging of the cartridge can be sufficient to prevent evaporation of the PCR reagents. The cartridge can be refrigerated until use. The collection device can comprise a plug on one end such that when inserted the plug can self lock into the cartridge, preventing inadvertent release of contaminants.

A user, for example, a customer, or investigator can push, or dispense, or place the cartridge into a cartridge receptacle in the system. Cells in the cartridge can be lysed in the system, for example, by heat, electro-poration, sonication, chemical, or mechanical tearing, rolling, beading-bashing, or other means of lysing. If heat is used, an electrode included in the cartridge can be used as a resistive heater by passing current through it.

Nucleic acids, and other charged molecules can be extracted from the lysis composition. Electrodes in the system can be turned on, and negatively and positively charged molecules can migrate to the appropriate electrodes. The molecules can pass through a sieving matrix and into the nucleic acid capture membrane where the nucleic acid can be captured by the pore walls. Large moieties such as cell debris can be blocked from entering into the nucleic acid capture membrane by the sieving matrix. Small moieties other than the nucleic acid, for example, proteins that may be PCR inhibitors, can pass through the sieving matrix and can also pass through the nucleic acid capture membrane to deposit onto the second electrode. The nucleic acid capture membrane can have specificity.

The direction of the current being applied to the electrode can be reversed such that the small moieties at the second electrode can be moved back though the nucleic acid capture membrane and sieving matrix into the sample receiving space. This can prevent the small moieties from inhibiting PCR.

Thermal cycling can be used for PCR. Resistive heating in the electrodes can produce the necessary heat for the PCR. The first electrode can receive a current that can create a temperature of about 60° C. to about 65° C. The second electrode can receive a current that can create a temperature of about 90° C. to about 95° C. The second electrode positioned next to the nucleic acid capture medium can quickly heat the capture membrane to about 90° C. to about 95° C., even when the first electrode can keep the remainder of the cartridge at a constant temperature of about 60° C. to about 65° C. In some embodiments, the cartridge can be used with proteins. The proteins can react with labeled antibodies.

A light source can be turned on to illuminate and/or excite any cleaved reporters in or on the nucleic acid capture membrane. The excited reporters can emit a light at a different wavelength than the illumination wavelength. At least some of the emitted light can pass through the second electrode and window, through lenses and into a photon detector such as a camera, or a photodiode. In various embodiments the fluorescence can be detected merely by eye. Detection of light can indicate the presence of a target matching the Taqman reagent sequence.

Various aspects of the preparation can require user intervention, while other aspects can be electronically controlled. Determination of user or electronically controlled intervention can be determined as deemed appropriate by one of skill in the art.

The method can provide one or more of the following:

1. Use of gold-standard Taqman without modifications;

2. Detection of a single pathogen (>10⁶ reporters in >40 nanoliter);

2. Simplicity, i.e., no sensor, no motors, no valves requiring only three (3) user steps;

4. Greater precision (counting) than other Taqman instruments;

5. Low-cost consumables and instrumentation;

6. Multiplexing by using probes with different colors.

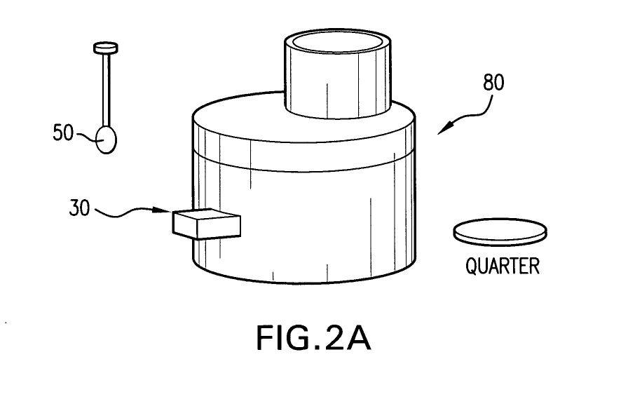

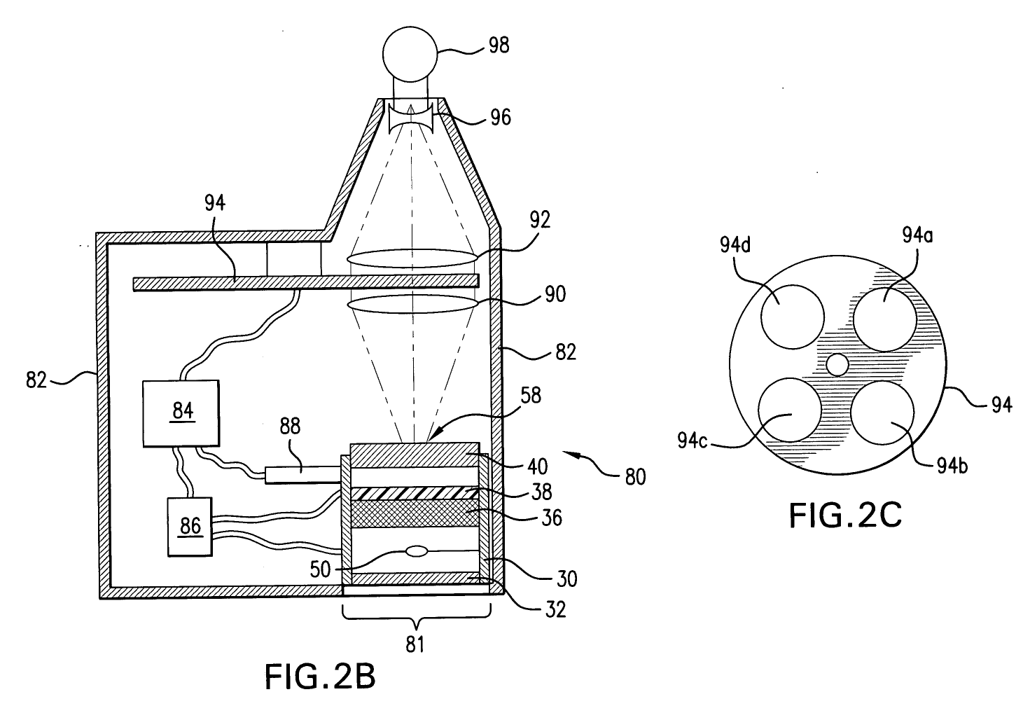

FIG. 2A is a perspective view of a cartridge processing device 80 that can use cartridge 30 illustrated in FIG. 1, and biological sample swab 50. For comparative purposes, FIG. 2A also depicts a U.S. Quarter for the purpose of showing the relative size of system 80 and cartridge 30 according to various embodiments. Cartridge processing device 80 can be a low-cost, hand-held, micro-device that uses nucleic acid chemistry to detect bacterial pathogens with high accuracy and specificity. For example, the nucleic acid chemistry can comprise Taqman chemistry. As illustrated in FIG. 2A, cartridge 30 is not much bigger than a quarter. Power can be provided to the electrodes that can be part of the cartridge that can use off-the-shelf batteries, for example, a 1-volt battery, a 1.5-volt battery, a 9-volt battery, or two AA 1.5 volt batteries.

According to some embodiments, and as illustrated, in FIG. 2B, cartridge 30 can be introduced into system 80. System 80 can comprise a housing 82. Housing 82 can comprise, for example, plastic, metal, glass, or a combination thereof. Housing 82 can comprise a receptacle 81, configured to interface with cartridge 30. Additional information concerning cartridge 30 can be found in the description of FIG. 1 herein. System 80 can comprise a control unit 84 disposed inside the housing. Control unit 84 can comprise a processor, for example a central processing unit, a digital signal processor, an analog to digital converter, or other suitable devices known to those skilled in the art. Control unit 84 can be electrically connected to a power source 86. Power source 86 can comprise a battery, a transformer connected to a wall outlet, or a combination thereof and the like. Power source 86 can be disposed inside housing 82. Control unit 84 can be electrically connected to excitation source 88 disposed inside housing 82. An excitation source can comprise one or more light emitting diodes, lasers, lamps, or combinations thereof and the like.

According to various embodiments, light from excitation source 88 can illuminate the capture membrane present in the cartridge. Reporter molecules present on the capture membrane can also be illuminated. Light can be emitted from the cartridge through transparent portion 56 illustrated in FIG. 1 of cartridge 30. Light emitted from the cartridge can be collected by a first lens 90 and further refracted by a second lens 92. System 80 can comprise filter wheel 94. Filter wheel 94 can be disposed between first lens 90 and second lens 92. As illustrated in FIG. 2C, filter wheel 94 can comprise different color filters, 94a, 94b, 94c and 94d combining red, green, blue or other color filtering, for example. Filter wheel 94 can rotate to position different filters between lenses 90 and 92. The filter wheel can be manually turned. In some embodiments, filter wheel 94 can be turned under the control of control unit 84 by a drive (not shown). Light emerging from second lens 92 can be further refracted by a third lens 96. A detection apparatus 98 can be disposed outside housing 82 in a position to collect light emerging from third lens 96. Alternatively, detection apparatus 98 can be disposed inside housing 82. Detection apparatus 98 can comprise, for example, a scanning photo detector, a charged coupled device, a digital Taqman™ Analyzer from Applied Biosystems, Foster City, Calif., a human eye, or any other suitable light detection apparatus.

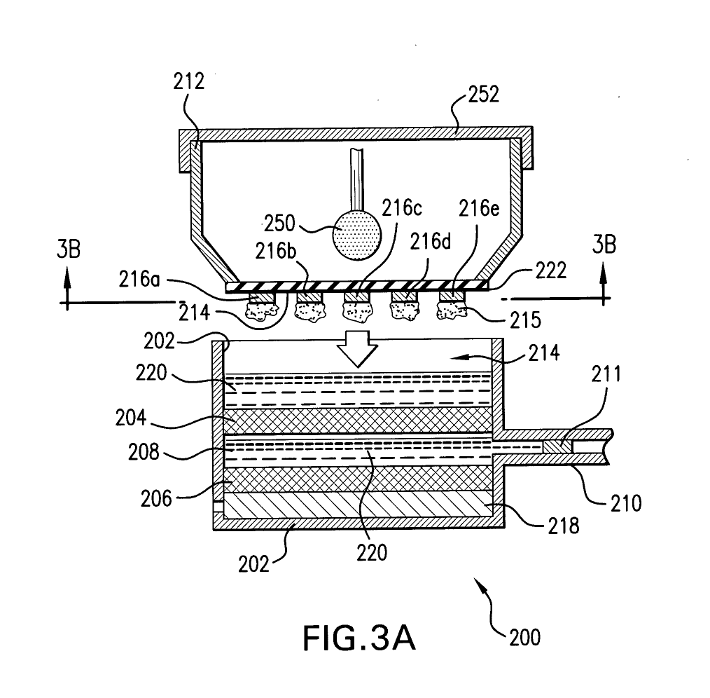

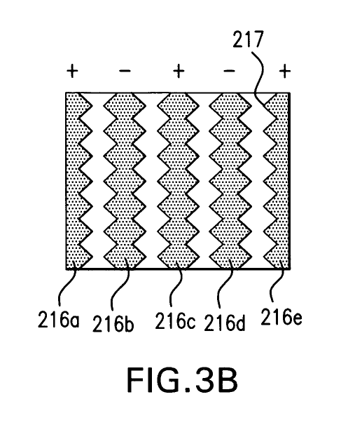

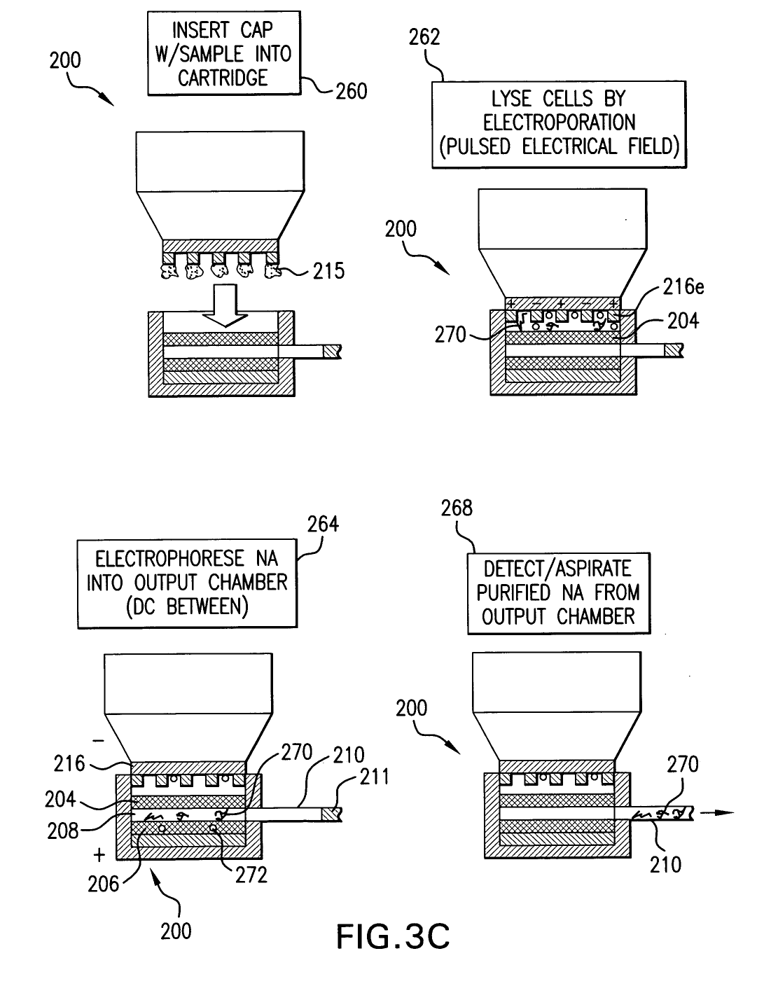

According to various embodiments, FIG. 3A illustrates a cartridge 200 that can comprise chamber 202. Chamber 202 can comprise one or more walls that define an interior space. A first sieving matrix 204 can be disposed in the interior space of chamber 202. First sieving matrix 204 can comprise, for example, a fiber filter, a micro-porous filter, a frit layer, a bead layer, a fiber composite layer, a laser drilled membrane, or any other material that can selectively allow nucleic acids to pass there through. A second sieving matrix 206 can be disposed inside chamber 202, adjacent to, but spaced apart from, first sieving matrix 204. Second sieving matrix 206 can comprise, a non-specific sequestering agent, for example, agarose, polyacrylamide, polyethylene glycol, or other conductive polymer. Cartridge 200 can be disposed in electrical contact with an electrical circuit (not shown) comprising at least a power source, a capacitor, a charging resistor, and a plurality of switches.

A collection area 208 can be defined between first 204 and second 206 sieving matrixes. Collection area 208 can be in fluid communication with a collection tube 210. Collection tube 210 can be disposed upon chamber 202. Collection tube 210 can be a capillary tube. A plug 211 can be disposed in collection tube 210. According to various embodiments, a plug can comprise, for example, a low melting point mixture of a high molecular weight compound that is solid at room temperature, for example, mineral wax, polyethylene glycol, and other suitable low melting point compounds known to one skilled in the art.

According to various embodiments, cartridge 200 can comprise a cap 212. Cap 212 can be configured to be inserted into chamber 202. Cap 212 can comprise a first electrode 216a, 216b, 216c, 216d, and/or 216e disposed at the bottom of cap 212. First electrode 216a, 216b, 216c, 216d, and 216e can comprise a plurality of electrodes.

For example, first electrode 216 can comprise electrodes 216a, 216b, 216c, 216d, and 216e arranged adjacent one another as shown in a linear manner in FIG. 3B. The arrangement of electrodes in first electrode 216 can be configured in a variety of ways so as to allow for the formation of field emission points 217 sufficient to permanently electroporate biological cells and/or viruses, for example, a zig-zag formation. First electrode 216a, 216b, 216c, 216d, and 216e can be shaping with a jagged edge pattern of ridges as shown for example in FIG. 3B. Field emission points 217 can enable high field strength between first electrode 216a, 216b, 216c, 216d, and 216e and a second electrode 218 disposed in chamber 202 adjacent second sieving matrix 206. This can allow for a much lower voltage to be utilized than if the electrodes were smooth or flat. During electroporation, electrical charge can be applied alternatively to the ridges such that the space between the ridges has a negative electrical charge in an electrode and a positive electrical charge on another adjacent electrode.

Cap 212 can enable the removal of first electrode 216a, 216b, 216c, 216d, and 216e from cartridge 200 and later reinsertion into cartridge 200. When reinserted, cap 212 can align parallel and geometrically close to second electrode 218. For example, first electrode 216a, 216b, 216c, 216d, and 216e can be separated from second electrode 218 by a few millimeters.

According to various embodiments and as illustrated in FIG. 3A, a biological sample 215 can be deposited into sample receiving space 214, by depositing sample 215 onto first electrode 216 (also see FIG. 3C), where it can be dried or adhered due to capillary forces. In some embodiments sample 215 can be deposited into sample receiving space 214. Cap 212 can be disposed upon chamber to form a fluid tight seal in an opening of chamber 202. When cap 212 is thus disposed it is in fluid contact with buffer 220.

According to some embodiments, a sample can be disposed directly upon first electrode 216, comprising a plurality of electrodes 216a-216e. For example, cap 212 can be pressed, or stamped against a biological sample, or brought into contact with a biological sample and then inserted into cartridge 200. The direct disposition of a biological sample can be advantageous for viscous biological samples, for example, sputum or fecal matter. In some embodiments sample 215 can be deposited into sample receiving space 214.

In some embodiments, cap 212 can comprise a reservoir, a lid 252, and a floor 222. Floor 222 can allow a liquid sample disposed in the reservoir, for example, from swab 250, to flow from the reservoir into sample receiving space 214.

According to some embodiments, cartridge 200 can comprise a buffer solution 220. A biological sample in buffer solution 220, can be irreversibly electroporated through the application of electric current pulses to first electrode 216. Electroporation can lyse cells or viruses in the biological sample. For example, a positive charge can be applied to electrodes 216a, 216c, and 216e, and a negative charge can be applied to electrodes 216b and 216d. An electric current can be provided at, for example, 110 volts with a resistance of, for example, about 40 ohms to about 70 ohms, or the like. The voltage pulses can be sufficient to irreversibly electroporate cells or viruses present in the sample. When electrodes 216a-216e are utilized in this manner, biological sample 215 can be electroporated.

According to various embodiments, when movement of polar analytes suspended in buffer 220 is desired, an electric field can be created between first electrode 216 and second electrode 218. The electric field can be sufficient to cause the migration of charged molecules between the first and second electrodes. The electric field can be applied for a time period sufficient to allow for the migration of the charged molecules into first sieving matrix 204, collection area 208, and second sieving matrix 206. A portion of the biological sample can be isolated in collection area 208. A portion of the biological sample present in collection area 208 can be removed from cartridge 200 via collection tube 210. Biological materials present in first sieving matrix 204, and/or present in second sieving matrix 206, can be retained in cartridge 200 during extraction.

FIG. 3C shows the operation steps. Reference in the figure to “NA” refers to nucleic acids. Additional details of various features referred to in the FIG. 3C can be found in FIG. 1, FIG. 2B, and FIG. 3A. In step 260, cap 212 can be used to collect biological sample 215 directly onto first electrode 216a, 216b, 216c, 216d, and 216e. Biological sample 215 can comprise cells. The cell can comprise nucleic acid. Cap 212 can then be snapped or disposed back into cartridge 200.

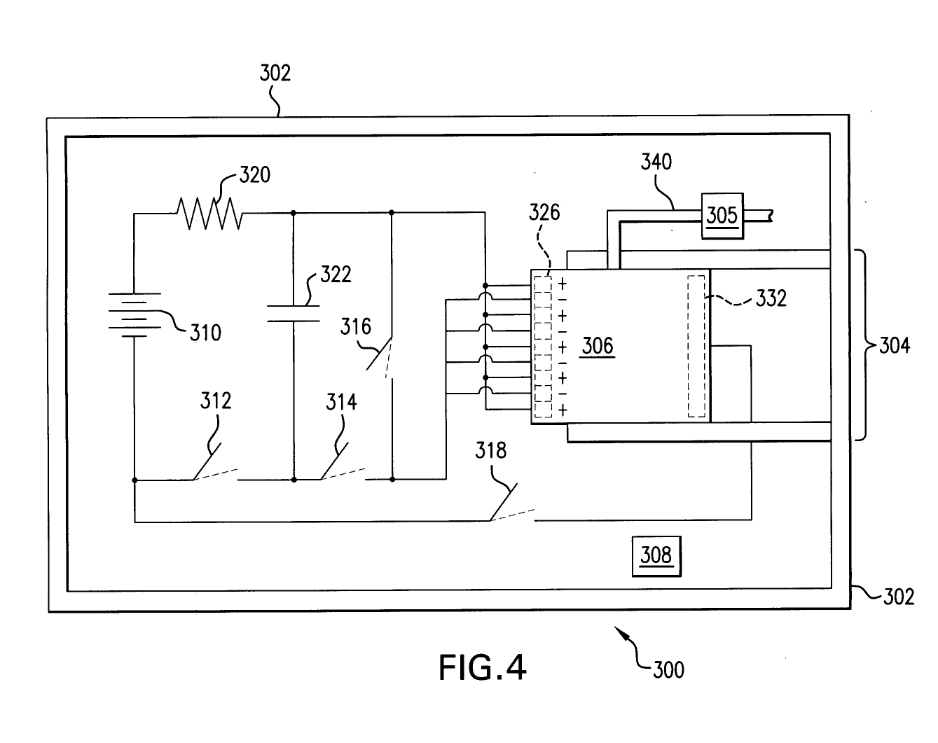

In step 262, a pulsed electrical field (PEF) can be generated between the ridges of first electrode 216a, 216b, 216c, 216d, and 216e to lyse cells in the buffer. With the cells lysed, nucleic acid 270 and impurities 272 can be released. The PEF can be formed with a capacitor charging circuit as illustrated in FIG. 4. In this circuit, switch 312 can be closed, and switches 314 and 316 can be open, thus charging capacitor 312 with power source 310 through charging resistor 322. After capacitor 312 has charged, switch 314 can be closed to release the current to discharge as an electrical pulse between the ridges of the plurality of electrodes 216a, 216b, 216c, 216d, and 216e in the first electrode.

In some embodiments as illustrated in FIG. 3C, electrophoresis can extract nucleic acid 270 from the lysis mixture or solution, through separation matrix 214 and into collection area 208. As seen in step 264, switch 316 and 318 (also see FIG. 4) can be closed and switches 312 and 314 can be opened, making first electrode 216a, 216b, 216c, 216d, and 216e negatively charged. Negatively charged first electrode 216a, 216b, 216c, 216d, and 216e can repulse the negatively charged nucleic acid 270. Simultaneously, second electrode 218 can be positively charged, attracting negatively charged nucleic acid 270. Positively charged molecules and/or impurities 272, for example, proteins, can be separated from nucleic acid 270 and collected at first electrode 216a, 216b, 216c, 216d, and 216e. Negatively charged molecules and/or impurities 272 can move along with nucleic acid 270. Negatively charged impurities 272 can be assumed to move faster or slower than nucleic acid 270. Electrophoresis can be timed or sensed to stop when nucleic acid 270 has moved to collection area 208. This timed or sensed stopping can trap negatively charged molecules and/or impurities 272 in one of first matrix 204 or second matrix 208.

In some embodiments, and as seen in step 268, collection area 208 can be pumped dry through collection tube 210. The pumping force can be provided by means known in the art, for example, by capillary force, by aspiration, or by a pump. If collection area 208 is emptied when nucleic acid 270 is in collection area 208, nucleic acid 270 can be extracted. Alternatively, a window (not shown) can be provided to collection area 208 and nucleic acid 270 can be amplified and/or detected in collection area 208.

According to various embodiments, an electrode can comprise a plurality of electrodes. Two or more pluralities of electrodes can be configured to form an electric field adapted to irreversibly electroporate biological material. A plurality of electrodes can have a linear shape. Each of the first plurality of electrodes can be arranged generally parallel to one other.

According to various embodiments, a system can comprise a first electrical lead can be electrically connected with a first subset of the plurality of electrodes. A second electrical lead can be electrically connected with a second subset of the first plurality of electrodes. A third electrical lead can be electrically connected with at least one second electrode. A capacitor can be electrically connected with the first and second leads. A resistor can beelectrically connected with the first electrode. In some embodiements, a control unit adapted to form a first electrical pole in the first subset and a second electrical pole in the second subset different from the first electrical pole can be provided.

According to various embodiments, FIG. 4 illustrates a cartridge processing device 300 configured to process a cartridge 306. Cartridge 306 can be similar to cartridge 200, illustrated in FIG. 3A. Cartridge processing device 300 can comprise a housing 302 defining an internal space. Housing 302 can comprise a receptacle 304. Receptacle 304 can be adapted to receive cartridge 306. Receptacle 304 can provide access to the internal space defined by housing 302.

Cartridge processing device 300 can comprise a control unit 308. Control unit 308 can comprise a central processing unit, a digital signal processor, an analog to digital converter, or other suitable devices know to those skilled in the art. Control unit 308 can be electrically connected to and/or control a plurality of different devices present in housing 302. Cartridge processing device 300 can comprise a power source 310, for example, a battery or a transformer connected to a wall outlet or the like. Power source 310 can be electrically connected to the various devices present in cartridge processing device 300. Cartridge processing device 300 can comprise a pump 305. Pump 305 can be in fluid communication with a collection tube 340 of cartridge 306.

Cartridge processing device 300 can comprise a resistor 320. Resistor 320 can provide a resistance of, for example, from about 40 ohms to about 70 ohms, or the like. Cartridge processing device 300 can comprise a capacitor 322. The capacitor can store a sufficient amount of electricity to irreversibly electroporate a cell or virus, for example, 2.5 kilovolts of electricity, or greater.

Cartridge processing device 300 can comprise one or more switches, for example, switch 312, switch 314, switch 316, and switch 318. Each switch can be electrically connected to power source 310. A switch can establish or break electrical connections as described below.

According to various embodiments, cartridge processing device 300 can be configured for cellular electroporation. One configuration for lysis comprises opening switches 316 and 318 and closing switches 312 and 314. The lysis configuration can produce and conduct high voltage pulses of electricity. Capacitor 322 can store and release the high voltage pulses of electricity. High voltage pulses can create a pulsed electric field in cartridge 306 by applying opposite charges on adjacent first electrodes 326 present in cartridge 306. For example, a positive charge can be applied to electrodes 216a, 216c, and 216d while a negative charge can be applied to electrodes 216b, and 216d. Resistor 320 can modulate the voltage, from power source 310, which can be delivered to capacitor 322.

According to various embodiments, cartridge processing device 300 can be configured for the electrophoretic separation of molecules in cartridge 306. Cartridge processing device 300 can be configured to produce an electric field across cartridge 306. The electric field can be produced by applying a first electric charge to a first electrode 326, and by applying an opposite electric charge to a second electrode 332. A configuration for producing an electric field gradient can comprise opening switches 312 and 314, and closing switches 316 and 318.

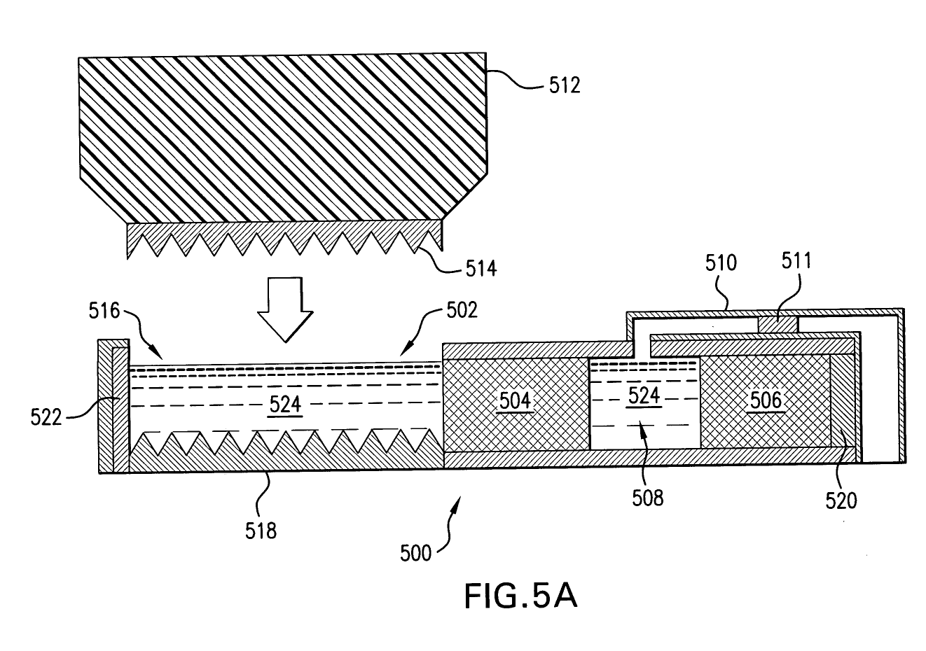

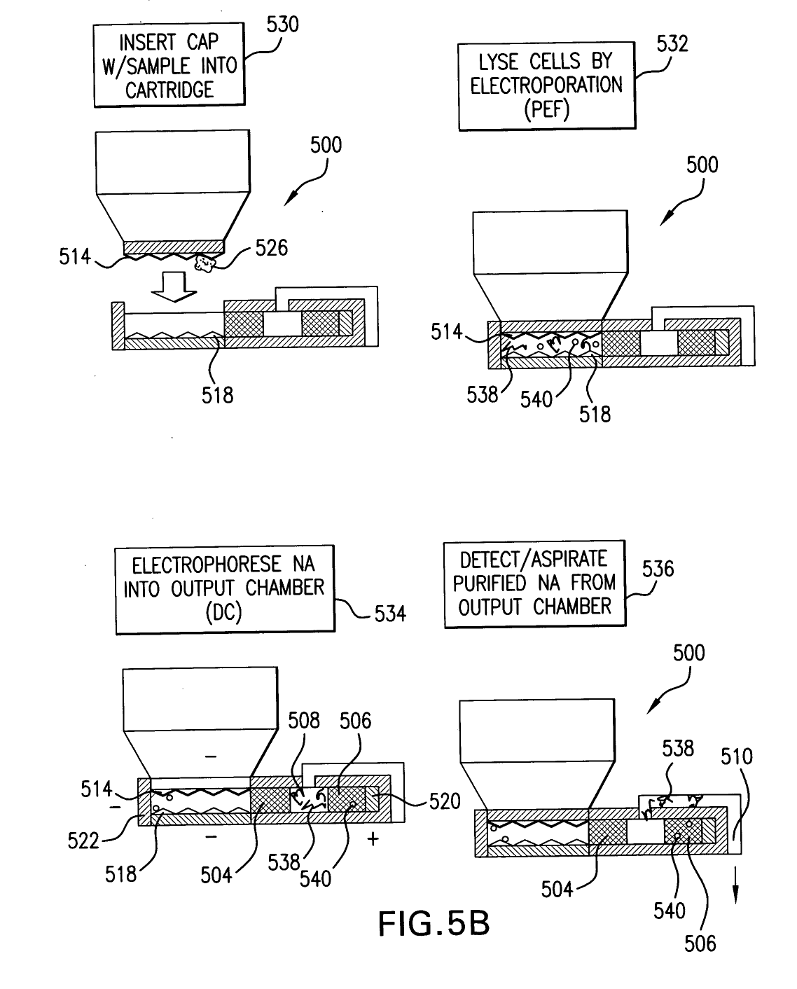

According to various embodiments, and as illustrated in FIG. 5A, a cartridge 500 can comprise a chamber 502 comprising an interior space. Cartridge 500 can comprise a first sieving matrix 504 disposed in the chamber 502. Cartridge 500 can comprise a second sieving matrix 506 disposed in the interior space of chamber 502. A collection area 508 can be defined in the chamber 502 between the first and second sieving matrixes 504 and 506. A collection tube 510 can be in fluid communication with collection area 508. Collection tube 510 can comprise a capillary tube. A plug 511 can be disposed in collection tube 510. Cartridge 500 can be loaded or preloaded with a buffer solution 524.

Cartridge 500 can comprise a cap 512. Cap 512 can comprise a first electrode 514. First electrode 514 can comprise a plurality of electrodes. Cap 512 can be configured to be secured in chamber 502. Chamber 502 can further comprise a second electrode 518 disposed at the end of cartridge opposite first electrode 514. A sample receiving space 516 can be defined between first electrode 514 and second electrode 518. Chamber 502 can further comprise a third electrode 520 and a forth electrode 522. Third electrode 520, space receiving area 516, first sieving matrix 504, collection area 508, second sieving matrix 506 and fourth electrode 520 can be arranged linearly, and/or sequentially, within the interior sample of chamber 502. This arrangement can allow a buffer 524 to be in liquid contact with third electrode 520, fourth electrode 522, and everything in between. First electrode 514 and second electrode 518 can be liquid contact with buffer 524.

Cap 512 can enable removal of first electrode 514 from cartridge 500 for deposit of a sample on first electrode 514 or direct-deposit into electrophoresis buffer 524. Cap 512 can later be reinserted into cartridge 500. When reinserted, cap 514 can be aligned parallel and spatially close to second electrode 518 with a few millimeters of buffer 524 between first electrode 514 and second electrode 518. Field emission points can be disposed in a surface or side of first electrode 514 facing second electrode 518. Field emission points can be disposed in a surface or side of second electrode 518 facing first electrode 514.

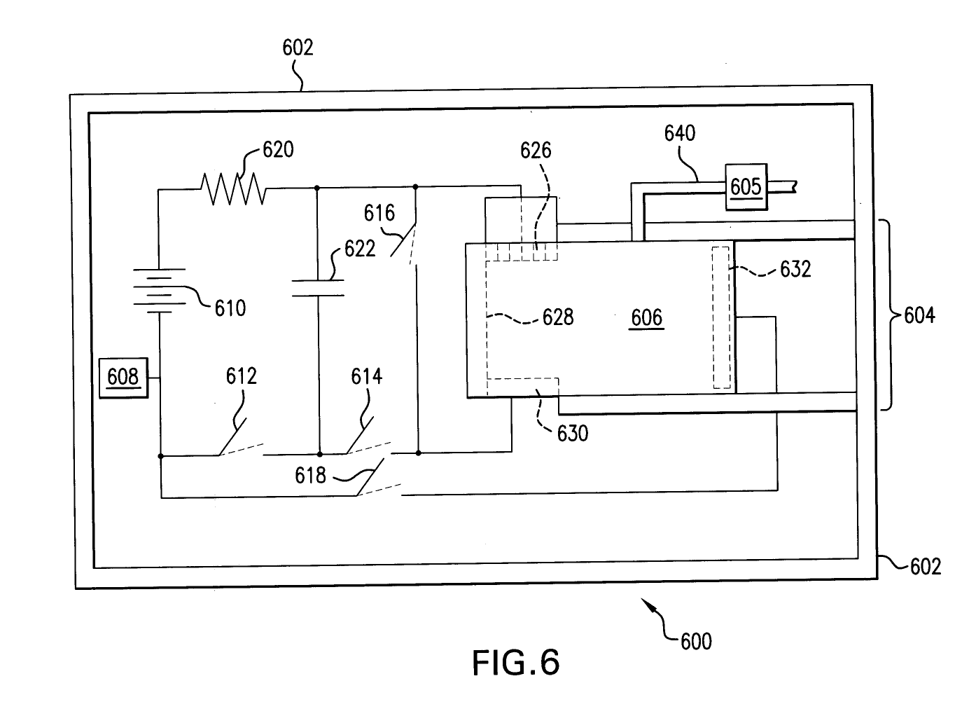

According to various embodiments, FIG. 5B shows a method of use for cartridge 500. Additional details of various features can be found in FIG. 5A. In step 530, cap 512 can be used to collect sample 526 directly onto first electrode 514 and can then snapped back into cartridge 500. In step 532, a pulsed electrical field (PEF) can be generated between first electrode 514 and second electrode 518, to lyse cells in the buffer. With the cells lysed, nucleic acid 538 and impurities 540 can be released. The PEF can be accomplished with a capacitor charging circuit of FIG. 6. In this circuit, switch 612 is closed and switches 614 and 616 are open, charging capacitor 622 with power source through 610 charging resistor 622. After capacitor 622 has charged, switch 614 can be closed releasing the current to quickly flow, for example, as an electrical pulse, from second electrode 518 to first electrode 514.

In some embodiments, step 534 can be used. Electrophoresis can extract nucleic acids 538 from the lysis mixture or solution, through first sieving matrix 504 and into collection area 508. Switch 616 and 618 are closed and, switches 612 and 614 are open, making first electrode 514, second electrode 518, and fourth electrode 522 negatively charged, repulsing any negatively charged nucleic acids 538. Simultaneously, third electrode 520 is positively charged, attracting nucleic acids 538. Positively charged impurities 540 (e.g. proteins) can be separated from nucleic acids 538 and collect at first electrode 514, second electrode 518, and fourth electrode 522. Negatively charged impurities 540 can move along with nucleic acids 538 but can be assumed to move faster or slower. Electrophoresis can be timed (or sensed) to stop when nucleic acids 538 are in collection area 508, then impurities 540 can be trapped in first matrix 504 or second matrix 506.

Step 536 illustrates nucleic acid 538 being pumped or detected in collection tube 510. Collection area 508 can be pumped dry through collection tube 510. When nucleic acids 538 are in collection area 508, nucleic acids 538 can also extracted.

In some embodiments, for example, cartridge 200 of FIG. 3A and cartridge 500 of FIG. 5A, the separation between respective electrodes of cartridge 200 and cartridge 500 can be about 600 μm. This can permit use of a low voltage power supply with cartridge 200 and cartridge 500.

According to various embodiments, FIG. 6 depicts a cartridge processing device 600 configured to process a cartridge 606. Cartridge 606 can be similar in design to cartridge 500 of FIG. 5A. Cartridge processing device 600 can comprise a housing 602 defining an internal space. Housing 602 can comprise a receptacle 604. Receptacle 604 can be adapted to receive cartridge 606. Receptacle 604 can provide access to the internal space defined by housing 602.

Cartridge processing device 600 can comprise a control unit 608. Control unit 608 can comprise a central processing unit, a digital signal processor, an analog to digital converter, or other suitable devices know to those skilled in the art. Control unit 608 can be electrically connected to and/or control a plurality of different devices present in housing 602. Cartridge processing device 600 can comprise a power source 610, for example, a battery or a transformer connected to alternating current, for example, a wall socket. Power source 610 can be electrically connected to the various devices present in cartridge processing device 600. Cartridge processing device 600 can comprise a pump 605. Pump 605 can be in fluid communication with a collection tube 640.

Cartridge processing device 600 can comprise a resistor 620. Resistor 620 can comprise a resistance of, for example, about 40 ohms, to about 70 ohms. Cartridge processing device 600 can comprise a capacitor 622. The capacitor can store electricity in the range of, for example, about 2.5 kilovolts of electricity, or greater.

Cartridge processing device 600 can comprise one or more switches, for example, switch 612, switch 614, switch 616, and switch 618. Each switch can be electrically connected to power source 610. The switches can establish or break electrical connections as described below.

According to various embodiments, cartridge processing device 600 can be configured for cellular electroporation. A configuration for electroporation can comprise opening switches 616 and 618 and closing switches 612 and 614. The configuration for electroporation can produce high voltage pulses of electricity. Capacitor 622 can store and release the high voltage pulses of electricity. The high voltage pulses can create a pulsed electric field in cartridge 606 by effecting opposite charges on a plurality of adjacent electrodes 626 present in cartridge 606.

According to various embodiments, electricity from power source 610 can be stored in capacitor 622. Switch 614 can be open in order to facilitate storing electricity in capacitor 622. Resistor 620 can modulate the voltage, from power source 610, which can be delivered to capacitor 622.

According to various embodiments, cartridge processing device 600 can be configured for electrophoresis of cartridge 606. Electrophoresis can be conducted by the production of an electric field gradient across cartridge 606. The electric field gradient can be produced by applying a first electric charge on a first electrode 628, and by applying an opposite electric charge on a second electrode 632. A configuration for producing an electric field gradient can comprise opening switches 612 and 614, and closing switches 616 and 618.

According to various embodiments, and as illustrated in FIG. 7, an apparatus can comprise a cartridge 700. Cartridge 700 can comprise a chamber 742. A first electrode 732 can be disposed adjacent to a wall inside cartridge 700. A second electrode 740 can be disposed adjacent a second wall inside cartridge 700. The first electrode 732 and second electrode 740 can be electrically connected to contacts 752 and 754 respectively. Contacts 752 and 754 can provide an electrical connection to the outside of cartridge 700.

Cartridge 700 can comprise an aperture 702. Aperture 702 can provide access to a sample receiving space 734 inside cartridge 700. Sample receiving space 734 can be defined between first electrode 732 and sieving matrix 736.

According to various embodiments, cartridge 700 can comprise a collection device 744. Collection device 744 can comprise a cap 746. Cap 746 can be configured to seal aperture 702 present in cartridge 700. Collection device 744 can comprise a sample collector 750, for collecting biological samples.

Once a biological sample has been loaded into cartridge 700, any intact cells or viruses can be lysed. Lysis can occur by electroporation of the biological sample. First electrode 732 and second electrode 740 can be configured to produce a pulsed electric field between them. Cartridge 700 can be loaded or pre-loaded with a buffer solution.

An electric charge can be applied to second electrode 740. An opposite electric charge can be applied to first electrode 732. An electric field gradient can be created between the first and second electrodes. The electric field gradient can attract or repel molecules in the sample receiving area depending on the charge of the molecules. For example, a positive charge on the second electrode can attract negatively charged molecules, for example, nucleic acids.

A collection chamber 730 can be defined in cartridge 700. Collection chamber 730 can be defined between sieving matrix 736, sieving matrix 740, and the walls of chamber 742. A sample extraction tube 748 can be in fluid communication with collection chamber 730. The sample extraction tube can comprise a capillary tube. A portion of a biological sample electrophoresed in cartridge 700 can be extracted in collection chamber 730 through sample extraction tube 748. The extraction of a portion of a biological sample can be timed to coincide with the arrival of the portion of the biological sample in collection chamber 730. Portions of the biological sample present in sieving matrix 736, sample receiving space 734, and sieving matrix 740 can be retained in cartridge 700 during the removal of a portion of the biological sample present in collection chamber 730.

In FIG. 7, a distance between electrodes 732 and 740 in cartridge 700 can be, for example, several millimeters, from 1 mm to about 10 mm, or about 6 mm. A voltage applied to electrodes 732 and 740 in cartridge 700 can be a function of the distance between electrodes 732 and 740.

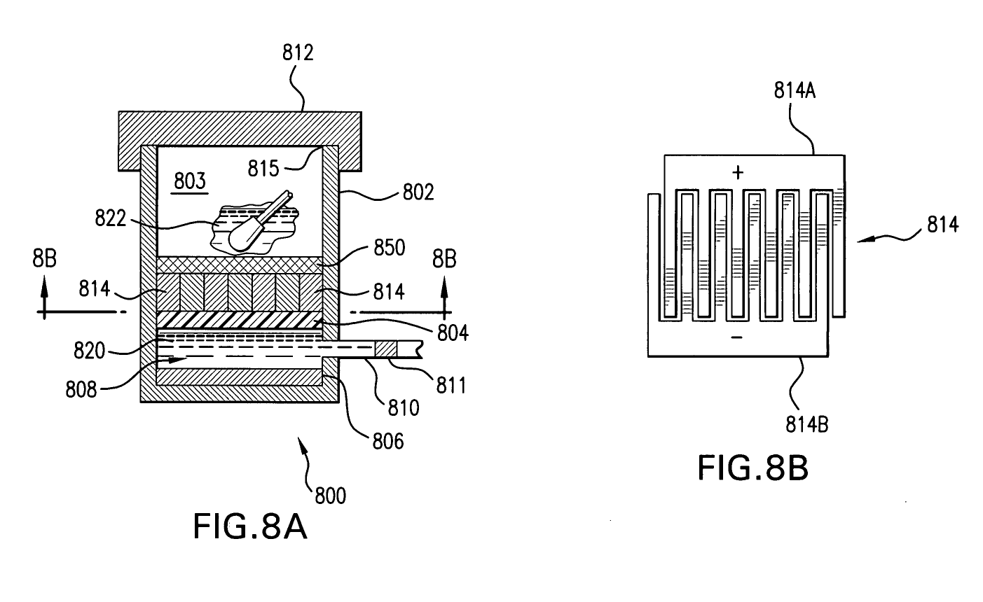

According to various embodiments, and as illustrated in FIG. 8A, the present teachings comprise a cartridge 800 comprising a chamber 802 having a first end defining an opening 815, and a second end. Chamber 802 can define an interior space 803. A swab comprising a sample fluid 822 can be disposed in interior space 803. Cartridge 800 can comprise a first sieving matrix 850, a porous membrane disposed in the interior space. First sieving matrix 850 can comprise, for example, a micro-porous filter, a laser drilled membrane, or any other material that selectively allows nucleic acids to passively diffuse therethrough.

According to various embodiments, cartridge 800 can comprise a first electrode 814. First electrode 814, as illustrated in FIG. 8B, can comprise two generally comb-shaped electrodes, 814A and 814B respectively. The comb-shaped electrodes can be interlaced with respect to each other. Electrodes can be any thickness, for example about 5 mm, about 4 mm, about 3 mm, about 2 mm, about 1 mm, about 0.5 mm, and the like. Electrodes 814A and 814B can be disposed adjacent one another in a generally interlaced format. Applying pulsed opposing electrical charges to electrodes 814A and 814B can result in the generation of pulsed electric fields sufficient to irreversibly electroporate biological cells and/or viruses.

Cartridge 800 can comprise a second sieving matrix 804 disposed in the interior space of chamber 802 adjacent, and generally parallel to, first electrode 814. Sieving matrix 804 can have any thickness, for example, about 2 mm, about 1 mm, about 0.5 mm, about 0.25 mm, and the like. Cartridge 800 can comprise a second electrode 806 disposed in the interior space of chamber 802. Second electrode can be disposed generally parallel to the first electrode.

A collection area 808 can be defined by or formed in the interior space between second sieving matrix 804 and second electrode 806. A buffer solution 820 can be disposed in collection area 808. The distance between second electrode 806 and sieving matrix 804 can be any distance, for example, 5 mm, 4 mm, 3 mm, 2 mm, 1 mm, 0.5 mm, and the like. An extraction tube 810 can be in fluid communication with collection area 808. Extraction tube 810 can comprise a capillary tube. A plug 811 can be disposed in capillary tube. Plug 811 can comprise, for example, a low melting point mixture of a high molecular weight compound that is solid at room temperature, for example, mineral wax, polyethylene glycol, and other suitable compounds know to one skilled in the art.

Cartridge 800 can be loaded with buffer solution 800. Cartridge 800 can comprise a cap 812. Cap 812 can be configured to attach to the first end chamber 802 and seal the opening.

In operation, sample fluid 822 can pass through first sieving matrix 850 and disperse through first electrode 814. According to various embodiments, sample fluid 822 can be electroporated by PEF and the electroporated sample fluid 822 can pass through second sieving matrix 804 and enter collection area 808. A pulsed electric field can be created by applying electric current to first electrode 814. The pulsed electric field can irreversibly electroporate or lyse any biological cells and/or viruses present in the biological sample. Nucleic acids in the biological sample can diffuse across the first sieving matrix 850. Other higher molecular weight biological materials in the biological sample can be prevented from diffusing across the membrane.

Opposite electrical charges can be applied to first 814 and second 806 electrodes to create an electric field gradient. The electric field gradient can induce the movement of nucleic acids present in cartridge 800 toward second electrode 806 and into collection area 808. Heat can be applied to plug 811 to melt plug 811. Nucleic acids can be drawn into extraction tube 810 by capillary forces or with a pump (not shown).

The various features of the cartridge can have any dimensions and configurations compatible with the utilities of the present teachings. In some embodiments, smaller dimensions for cartridges, electrodes, and separation matrices can be utilized in order to facilitate high sample throughout. For example, the cartridges can have any of a variety of cross-sectional configurations, such as square, rectangular, semicircular, circular, concave, or V-shaped, with a broad range of widths and depths. The cartridges can have rectangular, square, or concave cross-sections with depths and widths usually from about 2 mm to 20 mm, from about 10 mm to about 50 mm. The length of the cartridge can be selected to permit a desired degree of separation of sample components, with shorter lengths providing shorter electrophoresis times at the expense of decreased separation, and longer lengths providing longer separation paths and greater separation at the expense of longer electrophoresis times. For example, cartridge lengths of from about one cm to about 50 cm lengths are suitable for many separations, although longer and shorter lengths can be used as well.

The collection areas can have any configuration such as circular, oval, square, rectangular, or the like. The sizes and configurations of the chambers linked to each microchannel can be the same or different. For example, the sample receiving area can be large enough to receive a sufficient sample volume, for example, about 10 μL or less, from about 10 to about 100 mL, or from about 100 mL to about 1 mL. More generally, it is the preferred that the entire chamber in the cartridge be large enough to contain a sufficient amount of buffer to avoid buffer depletion during electrophoresis.

The electrodes for generating electrical currents can be made of any suitable conductive material, and are typically made from one or more metals or alloys. Exemplary electrode materials include copper, silver, platinum, palladium, carbon, nichrome, and gold. The electrode materials can be formed by known methods, conveniently by vapor deposition, silkscreen imprint, or other patterning techniques. The electrode materials may be coated with appropriate coating materials to inhibit electrochemical reactions with samples and reagents. For example, electrodes may be coated with a permeation layer having a low molecular weight cutoff that allows passage of small ions but not reagent or analyte molecules, as described, for example, in PCT Publ. Nos. WO 95/12808 and WO 96/01836.

The cartridge can be formed from any material, or combination of materials, suitable for the purposes of the present teachings. Materials that can be used include various plastic polymers and copolymers, such as polypropylenes, polystyrenes, polyimides, and polycarbonates. Inorganic materials such as glass and silicon are also useful. Silicon is advantageous in view of its compatibility with microfabrication techniques and its high thermal conductivity, which facilitates rapid heating and cooling of the cartridge, if necessary.

Sample components of interest can be detected in the cartridges by any of a variety of techniques, such as fluorescence detection, chemiluminescence detection, UV-visible adsorption, radioisotope detection, electrochemical detection, and biosensors, for example. For optically based detection methods, for example, fluorescence, absorbance, or chemiluminescence, the cartridge can contain at least one detection zone.

Optical signals to be detected can involve absorbance or emission of light having a wavelength between about 180 nm (ultraviolet) and about 50 nm (far infrared) More typically, the wavelength is between about 200 nm (ultraviolet) and about 800 nm (near infrared). For fluorescence detection, any opaque substrate material in the zone of detection can exhibit low reflectance properties so that reflection of the illuminating light back towards the detector can be minimized. Conversely, a high reflectance can be desirable for detection based on light absorption. With chemiluminescence detection, where light of a distinctive wavelength is typically generated without illuminating the sample with an outside light source, the absorptive and reflective properties of the substrate assembly can be less important, provided that at least one optically transparent window is present for detecting the signal. All of the cartridge body can be assembly is transparent, to allow visualization of the entire cartridge.

The sample components or analytes to be measured can be labeled to facilitate sensitive and accurate detection. Labels can be direct labels which themselves are detectable or indirect labels that are detectable in combination with other agents. Exemplary direct labels include, for example, fluorophores, chromophores, (for example, ³²P, ³⁵S, ³H) spin-labels, chemiluminescent labels, dioxetane-producing moieties, radioisotopes, or nano-probes. Exemplary indirect labels can include enzymes that catalyze a signal-producing event, and ligands, for example, an antigen or biotin that can bind specifically with high affinity to a detectable anti-ligand, such as a labeled antibody or avidin.

Characteristics of various embodiments can comprise one or more of the following: no moving parts; compact size enables close stacking of multiple modules; the device can isolate nucleic acid from cell debris and PCR inhibitors; the device can be fully integrated and contained such that once a sample goes in it never comes out; the device can concentrate nucleic acid from milliliters of sample to microliters of product; nucleic acid can be suspended in PCR buffer and can be ready for amplification; the device can be a low-cost low throwaway consumable; size can be used to separate genomes, such that shorter nucleic acid sequences can be made to pass through the matrix, whereas longer sequences be prevented from passing through the device; can work with many sample types, including swabs, blood, sputum, feces, tissue; and the device can be incorporated into a portable diagnostic unit.

Other embodiments of the present teachings will be apparent to those skilled in the art from consideration of the present specification and practice of the present teachings disclosed herein. It is intended that the present specification and examples be considered as exemplary only.

What is claimed is:

1. A cartridge comprising:
a chamber having a first end and a second end;
a first electrode disposed adjacent the first end;
a second electrode disposed adjacent the second end;
a sample receiving area defined in the chamber between the first electrode and the second electrode;
a nucleic acid capture membrane disposed inside the chamber between the first electrode and the second electrode; and
a sieving matrix disposed inside the chamber between the sample receiving area and the nucleic acid capture membrane;
wherein the first electrode and the second electrode are configured to produce an electric field that extends from the first electrode to the second electrode and the second electrode comprises a portion transparent to visible light.

2. The cartridge of claim 1, further comprising a third electrode and a fourth electrode adapted to electrically contact a buffer disposed in the same receiving space.

3. The cartridge of claim 1, wherein the sieving matrix comprises a micro-porous filter, a frit layer, a bead layer, a fiber composite layer, or a laser drilled membrane.

4. The cartridge of claim 1, further comprising a collection device comprising a cap, wherein the chamber further comprises an opening in fluid communication with the sample receiving area, and the cap is adapted to seal the opening.

5. The cartridge of claim 1, further comprising nucleic acid amplification reagents disposed at least in the capture membrane.

6. The cartridge of claim 1, wherein at least one of the first and the second electrodes is configured to function as a resistive heater.

7. The cartridge of claim 6, further comprising a control unit adapted to use the resistive heater for thermal cycling a sample disposed in the sample receiving area.

8. A system comprising:
the cartridge of claim 1; and
a cartridge processing device, the cartridge processing device comprising: a housing, a receptacle formed in the housing and adapted to receive the cartridge, an excitation source adapted to illuminate components in the chamber, a detection device adapted to comprise a field of view of at least a portion of the chamber, and a first electrical contact and a second electrical contact adapted to form electrical communications with the first and second electrodes, respectively.

9. The system of claim 8, wherein the detection device comprises a detection window disposed in the housing.

10. The system of claim 8, wherein the detection device comprises one or more optical filters disposed in the housing, wherein the one or more optical filters are adapted to filter light emitted from the cartridge.

11. The system of claim 8, wherein the detection device comprises one or more lenses disposed in the housing, wherein the one or more lenses are adapted to refract light emitted from the cartridge.

12. The system of claim 8, wherein the cartridge processing device further comprises a power source adapted to provide electrical current to the first and second electrical contacts. wherein the electrical current is configured to form a pulse electric field between the first electrode and the second electrode in a desired pattern.

13. The system of claim 12, further comprising a control unit adapted to control the power supply to generate a direct current between the first electrode and the second electrode, when a buffer is disposed in the cartridge.

14. A cartridge comprising:
a chamber having a first end, a second end, a side wall, and an opening defined in the first end;
a cap disposed in the opening, the cap comprising at least one first electrode;
at least one second electrode disposed at the second end of the chamber;
a first sieving matrix disposed in the chamber between the cap and the at least one second electrode; and
a second sieving matrix disposed in the chamber between the first sieving matrix and the at least one second electrode, the first and second sieving matrixes being spaced from one another to provide a collection area disposed in the chamber between the first sieving matrix and the second sieving matrix.

15. The cartridge of claim 14, wherein the first sieving matrix comprises a micro-porous filter, a frit layer, a bead layer, a fiber composite layer, a cellulose acetate membrane, a porous-polymer membrane, or a laser drilled membrane, and the second sieving matrix comprises agarose, polyethylene, or acrylamide.

16. A method of sample preparation, comprising:
loading a biological sample into a cartridge, the cartridge comprising first and second ends, at least one first electrode at the first end, at least one second electrode at the second end, a first sieving matrix disposed between the at least one first electrode and the at least one second electrode, a second sieving matrix disposed between the first sieving matrix and the at least one second electrode and spaced apart from the first sieving matrix;
electroporating the sample by applying a voltage to the at least one first electrode and the at least one second electrode;
moving polar analytes in the sample into the first sieving matrix by an electrically created motive force; and
collecting a portion of the sample resulting from the electrophoretic moving.

17. The method of claim 16, further comprising lysing the sample by chemical lysing, heating, or sonicating the sample.

18. The method of claim 16, wherein the collecting comprises removing a portion of the sample from the cartridge.

19. The method of claim 16, wherein the detecting comprises visually detecting the emission beams.

20. The method of claim 16, further comprising thermal cycling the biological sample.
